(19) United States
(12) Patent Application Publication
(10) Pub. No.: US 2004/0138725 A1
(43) Pub. Date: Jul. 15, 2004

(54) Harmless wireless energy transmission to implant

(76) Inventor: Peter Forsell, Zug (CH)

Correspondence Address: NIXON & VANDERHYE, PC, 1100 N GLEBE ROAD, 8TH FLOOR, ARLINGTON, VA, 22201-4714, US

(21) Appl. No.: 10/665,367

(22) Filed: Jan. 28, 2004

Related U.S. Application Data
(60) Provisional application No. 60/412,014, filed on Sep. 20, 2002.

(51) Int. Cl. A61N 1/08

(52) U.S. Cl. 607/61

(57) ABSTRACT

A transmitter transmits an alternating magnetic field to a receiver implanted in a human's body to supply energy drawn from the alternating magnetic field to an energy consuming implant in the human's body. The transmitter comprises a coil adapted to generate the alternating magnetic field in a desired direction towards the implanted receiver. A shield shields an operator's hand from the alternating magnetic field generated by the coil. The shield includes a magnetizable core extending in the coil and a magnetizable casing integrated with the core and surrounding the rear end of the coil and the circumference of the coil along at least a portion of the longitudinal extension of the coil.

### CROSS REFERENCE TO RELATED APPLICATION

This application is based upon provisional application Serial No. 60/412,014 filed Sep. 20, 2002, the disclosure of which is hereby incorporated by reference herein.

### FIELD OF THE INVENTION

The present invention relates to a transmitter for transmission of an alternating magnetic field to a receiver implanted in a human's or animal's body to supply energy drawn from the alternating magnetic field to an energy consuming implant in the human's or animal's body.

### BACKGROUND

Such a transmitter typically includes a coil that wirelessly co-operates with a coil of the implanted receiver. It has been found, however, that it is very unpleasant to a person to operate a handheld transmitter, because bad sensations occur in the person's hand while the transmitter is transmitting the alternating magnetic field. These bad sensations were not reduced significantly when the transmitter was encapsulated in a plastic case with a certain distance between the hand and the coil. Probably, the alternating magnetic field somehow unfavorably influences the nerves of the hand. It is not possible to shield the hand from the magnetic field by providing a steel shield between the hand and the coil, because such a steel shield would shorten the magnetic field. Because of the above-noted problems, prior handheld transmitters of coil-to-coil type have not been commercialized.

### SUMMARY OF THE INVENTION

The object of the present invention is to provide an alternating magnetic field transmitter, which at least significantly reduces or even prevents unpleasant sensations to a person's hand during manual operation of the transmitter.

Another object of the invention is to provide an apparatus for wireless transfer of energy including a magnetic field transmitter, which at least significantly reduces or even prevents unpleasant sensations to a person's hand during manual operation of the transmitter.

Another object of the invention is to provide an apparatus for wireless transfer of energy including a magnetic field transmitter, which at least significantly reduces or even prevents environmental disturbances during operation of the transmitter.

Yet another object of the invention is to provide a method for harmless wireless transfer of energy to an energy consuming medical device implanted in a human's or animal's body.

Accordingly, in accordance with a first aspect of the present invention the invention provides a transmitter for transmitting an alternating magnetic field to a receiver, which is implanted in a human's or animal's body to supply energy drawn from the alternating magnetic field to an energy consuming implant in the human's or animal's body. The transmitter comprises a coil adapted to generate the alternating magnetic field in a desired direction towards the implanted receiver, the coil having a longitudinal extension, a front end to be directed towards the receiver and a rear end to be directed away from the receiver. The transmitter further comprises a shield adapted to shield the environment, in particular an operator's hand holding the transmitter, from the alternating magnetic field generated by the coil except at the front end of the coil. The shield includes a magnetizable core extending in the coil and a magnetizable casing integrated with the core and surrounding the rear end of the coil and the circumference of the coil along at least a portion of the longitudinal extension of the coil.

The shield reduces the peaks or transients of the alternating magnetic field and the magnetic field itself in the direction towards, for example, an operator's hand while the transmission efficiency of the transmitter in the opposite direction, i.e. the direction towards the implanted receiver, is maintained. As a result, the unpleasant hand sensations previously experienced by operators testing prior hand-held transmitters are practically eliminated by the transmitter of the invention. This improvement of the transmitter of the invention is of great significance, because the transmitter is intended for use daily by an operator, such as a doctor or nurse, to treat different patients with energy consuming implants, and a doctor or nurse would be very reluctant to use a transmitter that gives rise to the unpleasant hand sensations discussed above.

The casing may completely surround the coil except the front end thereof and the core may wholly extend along the longitudinal extension of the coil.

Alternatively, the casing may surround the circumference of the coil along a portion of the longitudinal extension of the coil and the core or/and coil may extend past the casing along the longitudinal extension of the coil, as seen in the direction towards the front end of the coil.

In accordance with an embodiment of the present invention, the casing includes a circular cylindrical wall and a circular gable wall joined to the cylindrical wall. The core extends centrally in the cylindrical wall from the gable wall and the coil is applied on the core with the rear end of the coil facing the gable wall. The cylindrical wall may be provided with cutouts, in order not to reduce the effective magnetic field too much. Alternatively, the cylindrical wall may be shorter than the core. For example, the cylindrical wall may extend axially from the gable wall along half the length of the core or a third of the length of the core. Depending on the strength of the transmitted magnetic field the length of the cylindrical wall may be more reduced or even eliminated. The coil may be shorter or longer than the core and/or the cylindrical wall.

In all embodiments of the invention, the shield preferably is made of ferrite or similar magnetizable material.

The transmitter suitably includes a plastic box, in which the coil and shield are arranged such that they are located at a distance, in the order of centimeters, from the operator's hand, when the operator holds the transmitter.

In accordance with a second aspect of the present invention, the invention also provides an apparatus for wireless transfer of energy from outside a human's or animal's body to an energy consuming medical device implanted in the human's or animal's body. The apparatus comprises a transmitter operable from outside the human's or animal's body for transmission of an alternating magnetic field, and a receiver implantable in the human's or animal's body for receiving the alternating magnetic field and for drawing energy from the alternating magnetic field to be supplied to the energy consuming implanted medical device. The transmitter includes at least one coil for generating the alternating magnetic field in a desired direction towards the receiver, wherein the coil has a longitudinal extension, a front end to be directed towards the receiver and a rear to be directed away from the receiver. The apparatus further comprises at least one shield adapted to shield the environment from the alternating magnetic field generated by the coil except at the front end of the coil. The shield includes a magnetizable core extending in the coil and a magnetizable casing integrated with the core and surrounding the rear end of the coil and the circumference of the coil along at least a portion of the longitudinal extension of the coil.

The transmitter of the apparatus may be designed in accordance with the embodiments described above in connection with the first aspect of the invention.

Alternatively, the apparatus may include two identical shielded transmitters that are to be placed at different sides of the implanted receiver, for example on the human's abdomen and back. The two transmitters may also be placed on a stand relative to the human's or animal's body so that the two transmitters are in the desired positions for transmitting the alternating magnetic fields towards the implanted receiver.

The apparatus may be used for supplying energy to implanted medical devices, such as adjustable restriction devices for treating obesity, heartburn and reflux disease, urinary and anal incontinence, infusion pumps, muscle stimulators, impotence or other wireless transfer of energy to implants.

In accordance with a third aspect of the present invention, there is provided a method for harmless wireless transfer of energy to an energy consuming medical device implanted in a human's or animal's body. The method comprises:

implanting in the human or animal a receiver capable of receiving an alternating magnetic field and of drawing energy from the alternating magnetic field to be supplied to the energy consuming medical device,

manually holding external to the body a transmitter capable of transmitting the alternating magnetic field, the transmitter including a coil for generating the alternating magnetic field, the coil having a longitudinal extension, a front end directed away from the hand holding the transmitter and a rear end facing the hand holding the transmitter,

transmitting by means of the transmitter the alternating magnetic field to the implanted receiver, and

shielding by means of a shield the hand holding the transmitter from the alternating magnetic field generated by the coil, wherein the shield includes a magnetizable core extending in the coil of the transmitter and a magnetizable casing integrated with the core of the transmitter and surrounding the rear end of the coil and the circumference of the coil along at least a portion of the longitudinal extension of the coil.

In a more general aspect, the present invention also provides another method for harmless wireless transfer of energy to an energy consuming medical device implanted in a human's or animal's body, comprising:

implanting in the human or animal a receiver capable of receiving an alternating magnetic field and of drawing energy from the alternating magnetic field to be supplied to the energy consuming medical device,

providing an external transmitter capable of transmitting the alternating magnetic field and including a coil for generating the alternating magnetic field, the coil having a longitudinal extension, a front end and a rear end;

positioning the transmitter relative to the body so that the front end of the coil is directed towards the receiver and the rear end of the coil is directed away from the receiver,

transmitting by means of the transmitter the alternating magnetic field to the implanted receiver, and

shielding by means of a shield the environment from the alternating magnetic field generated by the coil except at the front end of the coil, wherein the shield includes a magnetizable core extending in the coil of the transmitter and a magnetizable casing integrated with the core of the transmitter and surrounding the rear end of the coil and the circumference of the coil along at least a portion of the longitudinal extension of the coil.

### BRIEF DESCRIPTION OF THE DRAWINGS

FIG. 1 schematically illustrates an apparatus of the present invention.

FIG. 2 is a front view of a coil and shield of a hand-held transmitter according to an embodiment of the invention.

FIG. 3 is a cross-section along the line III-III in FIG. 2.

FIG. 4 is a perspective view of a coil and shield of a hand-held transmitter according to another embodiment of the invention.

FIG. 5 is a cross-section through the shield shown in FIG. 4.

FIG. 6 illustrates an embodiment of the apparatus of the invention used for transferring energy to an artificial sphincter applied on the rectum of an anal incontinent human being.

FIG. 7 is a modification of the embodiment according to FIG. 6.

### DETAILED DESCRIPTION OF THE INVENTION

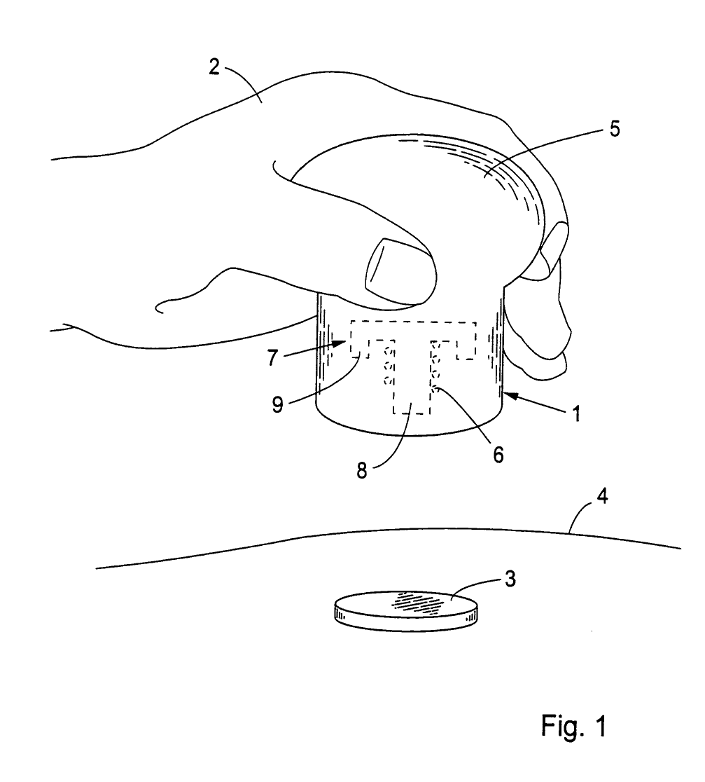

FIG. 1 illustrates manual operation of an apparatus of the invention including an alternating magnetic field transmitter 1 held by an operator's hand 2 and a receiver 3 subcutaneously implanted in a human's body 4. The implanted receiver 3 is capable of receiving the alternating magnetic field transmitted by the transmitter 1 and of drawing energy from the alternating magnetic field to be supplied to an energy consuming medical device implanted in the body 4. The transmitter 1 includes a plastic box 5 containing a coil 6 adapted to generate an alternating magnetic field in the direction away from the hand 2 towards the implanted receiver 3. The coil 6 is situated in the lower part of the box 5 a few centimeters from the hand 2.

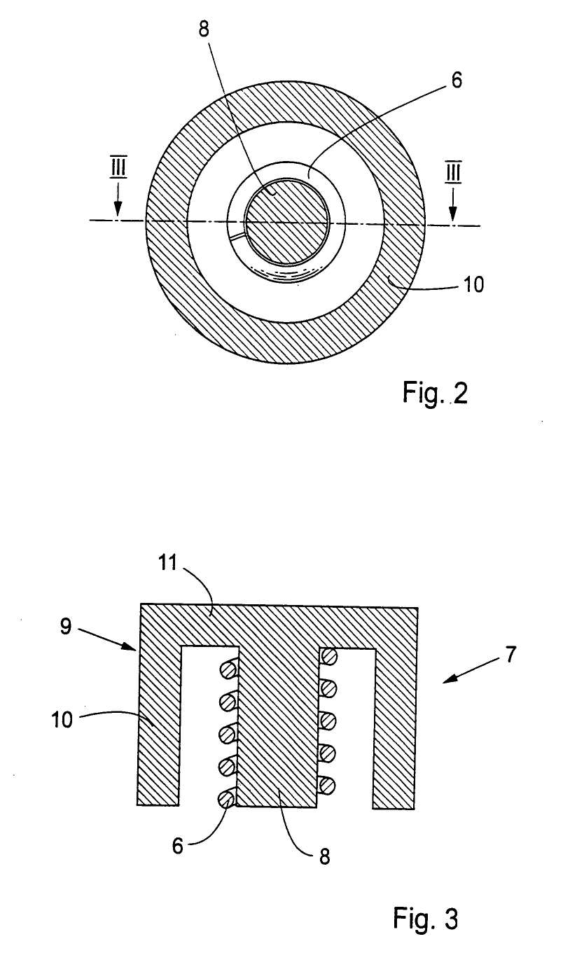

FIGS. 2 and 3 show a shield 7 of the apparatus for shielding the hand 2 from the alternating magnetic field including a ferrite core 8 extending in the coil 6 along the entire longitudinal extension thereof. (Alternatively, the coil 6 may be shorter than the core 8.) A ferrite casing 9 is integrated with the core 8 and surrounds the top rear end of the coil 6 and the circumference of the coil 6 along the entire longitudinal extension of the coil 6. The casing 9 includes a circular cylindrical wall 10 surrounding the coil 6 and a circular gable wall 11 joined to the cylindrical wall 10.

In FIG. 1 the shield 7 has a somewhat modified design, the position of the shield 7 being indicated in dotted lines. Thus, the cylindrical wall 10 extends only halfway along the coil 6.

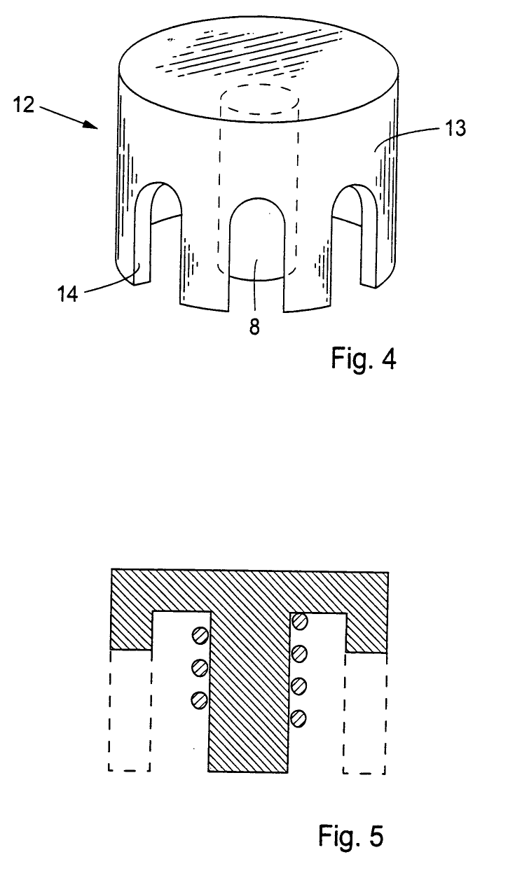

FIGS. 4 and 5 show a modified shield 12 that does not dampen the alternating magnetic field as much as the shield 7 of FIGS. 2 and 3. Thus, the cylindrical wall 13 of the shield 12 is provided with several cutouts 14 evenly distributed around the circumference of the cylindrical wall 13. In this embodiment, the coil 6 is shorter than the core 8.

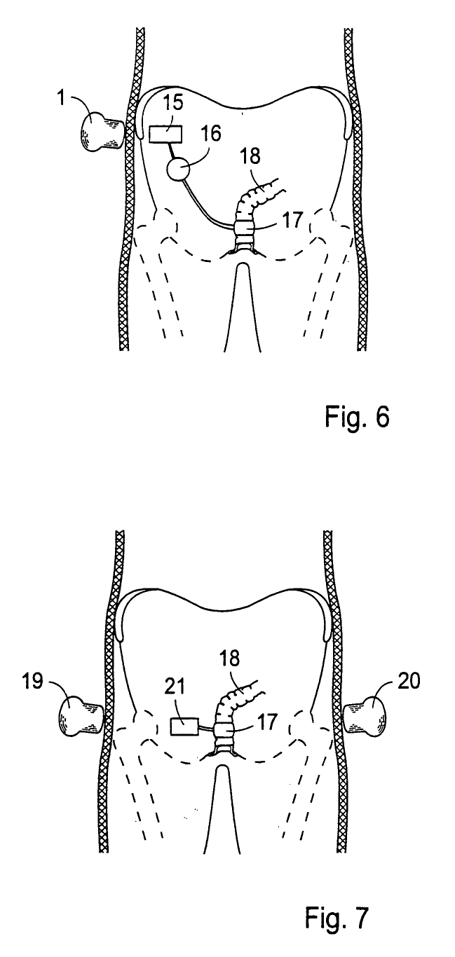

FIG. 6 shows an embodiment of the apparatus used for an anal incontinent human. The apparatus includes the transmitter 1 and a receiver 15 subcutaneously implanted in the human's body. The receiver 15 supplies energy to an implanted operation device 16 that operates an artificial sphincter 17 applied on the human's rectum 18. The transmitter 1 is held by an operator who puts it on the human's skin substantially in front of the implanted receiver 15 to provide efficient energy transmission from the transmitter to the receiver 15.

The embodiment shown in FIG. 7 is identical to the embodiment shown in FIG. 6, except that the apparatus includes a further wireless energy transmitter 19 and the receiver and operation device are integrated in a single receiver/operation unit 20 located close to the artificial sphincter 17. In this embodiment the transmitters 1 and 19 are positioned at different sides of the implanted receiver/operation unit 20, suitably at opposite sides as illustrated in FIG. 7, to transmit wireless energy to the receiver/operation unit 20.

Of course, two energy transmitters may also be used for transmitting energy to the receiver 15 of the embodiment shown in FIG. 6.

What is claimed is:

1. A transmitter for transmitting an alternating magnetic field to a receiver, which is implanted in a human's or animal's body to supply energy drawn from the alternating magnetic field to an energy consuming implant in the human's or animal's body, the transmitter comprising:
a coil adapted to generate the alternating magnetic field in a desired direction towards the implanted receiver, said coil having a longitudinal extension, a front end to be directed towards the receiver and a rear end to be directed away from the receiver, and
a shield adapted to shield the environment from the alternating magnetic field generated by said coil except at said front end of said coil, said shield including a magnetizable core extending in said coil and a magnetizable casing integrated with said core and surrounding said rear end of said coil and the circumference of said coil along at least a portion of said longitudinal extension of said coil.

2. A transmitter according to claim 1, wherein said casing completely surrounds said coil except said front end thereof.

3. A transmitter according to claim 2, wherein said core wholly extends along said longitudinal extension of said coil.

4. A transmitter according to claim 1, wherein said casing surrounds the circumference of said coil along a portion of said longitudinal extension of said coil.

5. A transmitter according to claim 4, wherein said core and/or coil extend past said casing along said longitudinal extension of said coil, as seen in the direction towards said front end of said coil.

6. A transmitter according to claim 1, wherein said casing comprises a circular cylindrical wall and a circular gable wall joined to said cylindrical wall, said core extends centrally in said cylindrical wall from said gable wall and said coil is applied on said core with said rear end of said coil facing said gable wall.

7. A transmitter according to claim 6, wherein said cylindrical wall is provided with cut-outs.

8. A transmitter according to claim 1, wherein said shield is made of ferrite.

9. A transmitter according to claim 1, further comprising a plastic box, in which said coil and shield are arranged such that they are located at a distance, in the order of centimeters, from an operator's hand, when the operator holds said transmitter during operation.

10. An apparatus for wireless transfer of energy from outside a human's or animal's body to an energy consuming medical device implanted in the human's or animal's body, comprising:
a transmitter operable from outside the human's or animal's body for transmission of an alternating magnetic field,
a receiver implantable in the human's or animal's body for receiving said alternating magnetic field and for drawing energy from said alternating magnetic field to be supplied to the energy consuming implanted medical device, said transmitter including at least one coil for generating said alternating magnetic field in a desired direction towards said receiver, said coil having a longitudinal extension, a front end to be directed towards said receiver and a rear end to be directed away from said receiver, and
at least one shield adapted to shield the environment from said alternating magnetic field generated by said coil except at said front end of said coil, said shield including a magnetizable core extending in said coil and a magnetizable casing integrated with said core and surrounding said rear end of said coil and the circumference of said coil along at least a portion of said longitudinal extension of said coil.

11. An apparatus according to claim 10, wherein said casing completely surrounds said coil except said front end thereof.

12. An apparatus according to claim 11, wherein said core wholly extends along said longitudinal extension of said coil.

13. An apparatus according to claim 10, wherein said casing surrounds the circumference of said coil along a portion of said longitudinal extension of said coil.

14. An apparatus according to claim 13, wherein said core and/or coil extends past said casing along said longitudinal extension of said coil, as seen in the direction towards said front end of said coil.

15. An apparatus according to claim 10, wherein said casing comprises a circular cylindrical wall and a circular gable wall joined to said cylindrical wall, said core extends centrally in said cylindrical wall from said gable wall and said coil is applied on said core with said rear end of said coil facing said gable wall.

16. An apparatus according to claim 15, wherein said cylindrical wall is provided with cut-outs.

17. An apparatus according to claim 10, wherein said shield is made of ferrite.

18. An apparatus according to claim 10, further comprising a plastic box, in which said coil and shield are arranged such that they are located at a distance, in the order of centimeters, from an operator's hand, when the operator holds said transmitter during operation.

19. An apparatus for wireless transfer of energy from outside a human's or animal's body to an energy consuming medical device implanted in the human's or animal's body, comprising:
a first transmitter operable from outside the human's or animal's body for transmission of a first alternating magnetic field,
a second transmitter operable from outside the human's or animal's body for transmission of a second alternating magnetic field,
a receiver implantable in the human's or animal's body for receiving and drawing energy from said first and second alternating magnetic fields to be supplied to the energy consuming implanted medical device, said first and second transmitters including first and second coils for generating said first and second alternating magnetic fields, respectively, in two different directions towards said receiver, each of said first and second coils having a longitudinal extension, a front end to be directed towards said receiver and a rear end to be directed away from said receiver, and
first and second shields adapted to shield the environment from said first and second alternating magnetic fields, each of said first and second shields including a core extending in the associated coil and a magnetizable casing integrated with said core and surrounding said rear end of said coil and the circumference of said coil along at least a portion of said longitudinal extension of said coil.

20. An apparatus according to claim 19, wherein each said casing of said first and second shields completely surrounds its coil except said front end thereof.

21. An apparatus according to claim 20, wherein each said core wholly extends along said longitudinal extension of its coil.

22. An apparatus according to claim 19, wherein each said casing surrounds the circumference of its coil along a portion of said longitudinal extension of said coil.

23. An apparatus according to claim 22, wherein each said core and/or coil extends past its associated casing along said longitudinal extension of its coil, as seen in the direction towards said front end of said coil.

24. An apparatus according to claim 19, wherein each said casing comprises a circular cylindrical wall and a circular gable wall joined to said cylindrical wall, said core associated with said casing extends centrally in said cylindrical wall from said gable wall and said coil associated with said casing is applied on said core with said rear end of said coil facing said gable wall.

25. An apparatus according to claim 24, wherein said cylindrical wall is provided with cut-outs.

26. An apparatus according to claim 19, wherein each shield is made of ferrite.

27. An apparatus according to claim 19, further comprising first and second plastic boxes, in which said first and second coils, respectively, and first and second shields, respectively, are arranged such that they are located at a distance, in the order of centimeters, from an operator's hand, when the operator holds said first or second transmitter during operation.

28. A method for harmless wireless transfer of energy to an energy consuming medical device implanted in a human's or animal's body, comprising:
implanting in the human or animal a receiver capable of receiving an alternating magnetic field and of drawing energy from the alternating magnetic field to be supplied to the energy consuming medical device,
manually holding external to the body a transmitter capable of transmitting the alternating magnetic field, the transmitter including a coil for generating the alternating field, the coil having a longitudinal extension, a front end directed away from the hand holding the transmitter and a rear end facing the hand holding the transmitter,
transmitting by means of the transmitter the alternating magnetic field to the implanted receiver, and
shielding by means of a shield the hand holding the transmitter from the alternating magnetic field generated by the coil, the shield including a magnetizable core extending in the coil of the transmitter and a magnetizable casing integrated with the core of the transmitter and surrounding the rear end of the coil and the circumference of the coil along at least a portion of the longitudinal extension of the coil.

29. A method for harmless wireless transfer of energy to an energy consuming medical device implanted in a human's or animal's body, comprising:
implanting in the human or animal a receiver capable of receiving an alternating magnetic field and of drawing energy from the alternating magnetic field to be supplied to the energy consuming medical device,
providing an external transmitter capable of transmitting the alternating magnetic field and including a coil for generating the alternating field, the coil having a longitudinal extension, a front end and a rear end,
positioning the transmitter relative to the body so that the front end of the coil is directed towards the receiver and the rear end of the coil is directed away from the receiver,
transmitting by means of the transmitter the alternating magnetic field to the implanted receiver, and
shielding by means of a shield the environment from the alternating magnetic field generated by the coil except at the front end of the coil, the shield including a magnetizable core extending in the coil of the transmitter and a magnetizable casing integrated with the core of the transmitter and surrounding the rear end of the coil and the circumference of the coil along at least a portion of the longitudinal extension of the coil.
